(19) United States
(12) Patent Application Publication
(10) Pub. No.: US 2018/0123053 A1
(43) Pub. Date: May 3, 2018

(54) BIPOLAR MATERIALS AND ORGANIC LIGHT EMITTING DIODES

(71) Applicant: Yuan Ze University, Chung-Li (TW)

(72) Inventors: Shu-Yun CHANG, Yilan City (TW); Guan-Ting LIN, Puyan Township (TW); Jau-Jiun HUANG, Kaohsiung City (TW); Man-kit LEUNG, Taipei City (TW); Tien-Lung CHIU, Taipei City (TW); Jiun-Haw LEE, Taipei City (TW); Chi-Feng LIN, New Taipei City (TW)

(73) Assignee: Yuan Ze University, Chung-Li (TW)

(21) Appl. No.: 15/801,822

(22) Filed: Nov. 2, 2017

(30) Foreign Application Priority Data
Nov. 3, 2016 (TW) 105135777

(51) Int. Cl. H01L 51/00; C09K 11/02; C07D 403/04

(52) U.S. Cl. CPC H01L 51/0072; H01L 51/5016; C07D 403/04; C09K 11/025

(57) ABSTRACT

A Carbazole-N-Benzimidazole bipolar material and organic light emitting diodes using the same is provided. The organic light emitting diodes include a substrate, a first conducting layer, a hole transporting layer, a light emitting layer, an electron transporting layer, and a second conducting layer. The first conducting layer is disposed on the substrate. The hole transporting layer is disposed on the first conducting layer. The light emitting layer having the Carbazole-N-Benzimidazole bipolar material is disposed on the hole transporting layer. The electron transporting layer is disposed on the light emitting layer. The second conducting layer is disposed on the electron transporting layer.

### BACKGROUND OF THE INVENTION

### 1. Field of the Invention

The present invention relates to bipolar materials for organic light emitting diodes. More particularly, the present invention relates to bipolar materials with connected Benzimidazole and Carbazole unit, and organic light emitting diodes using the bipolar materials.

### 2. Description of the Prior Art

Liquid crystal displays have become the mainstream of displaying devices in recent years. For example, televisions, computers, laptops, monitors, cell phones, and digital cameras, are electronic products using liquid crystal displays. A backlight module in a liquid crystal display is for providing sufficient brightness and uniform distribution of light so that the liquid crystal display may display images normally.

Organic light emitting diodes have gradually become the common light emitting units in view of their advantages of wide visual angle, short responding time, low energy consumption, and wide range of operation temperature. Recently, host-guest light emitting two-body systems are generally used by organic light emitting diodes, wherein a 100% internal quantum efficiency could be achieved theoretically by selecting appropriate phosphor guest emitter. Bipolar host materials having higher triplet energy gap and good electron and hole transporting properties would increase the efficiency and lifetime of organic light emitting diodes.

### SUMMARY OF THE INVENTION

One object of the present invention is to provide a bipolar material having higher triplet energy gap and good electron and hole transporting properties.

Another object of the present invention is to provide an organic light emitting diode having better efficiency and longer lifetime.

The bipolar material of the present invention is selected from the group consisting of one of formula (1) to formula (4) or combinations thereof, wherein:

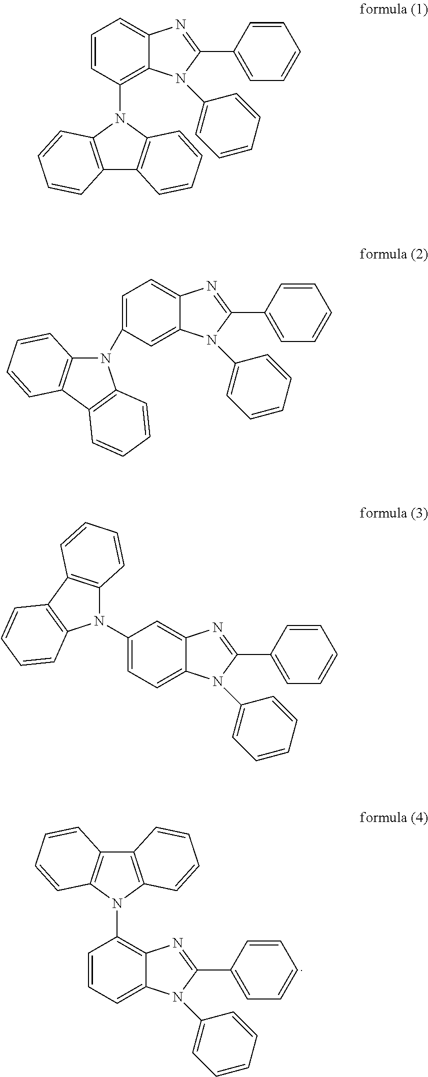 [SMILES: c1ccc(-c2nc3cccc(-n4c5ccccc5c5ccccc54)c3n2-c2ccccc2)cc1 ; c1ccc(-c2nc3ccc(-n4c5ccccc5c5ccccc54)cc3n2-c2ccccc2)cc1 ; c1ccc(-c2nc3cc(-n4c5ccccc5c5ccccc54)ccc3n2-c2ccccc2)cc1 ; c1ccc(-c2nc3c(-n4c5ccccc5c5ccccc54)cccc3n2-c2ccccc2)cc1]

The organic light emitting diode of the present invention includes a substrate, a first conducting layer, a hole transporting layer, a light emitting layer, an electron transporting layer, and a second conducting layer. The first conducting layer is disposed on the substrate. The hole transporting layer is disposed on the first conducting layer. The light emitting layer is disposed on the hole transporting layer. The light emitting layer includes a bipolar material selected from the group consisting of one of formula (1) to formula (4) or combinations thereof, wherein:

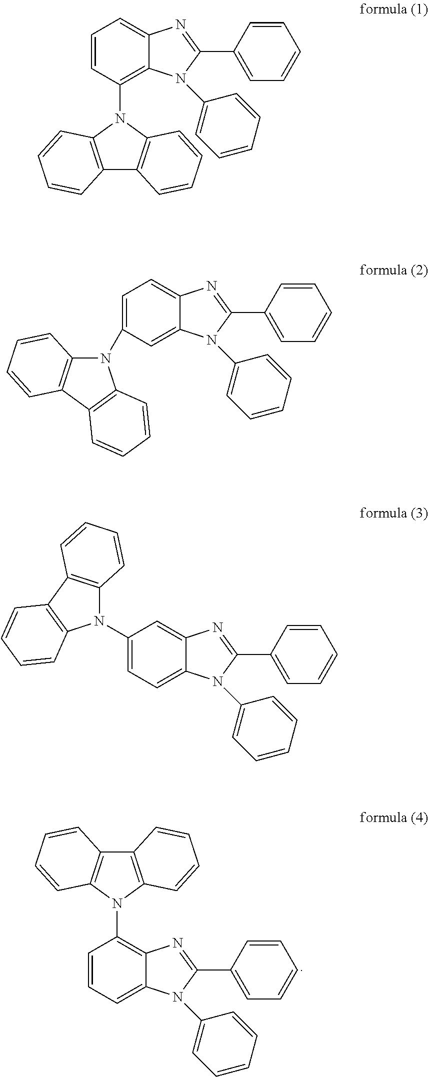 [SMILES: c1ccc(-c2nc3cccc(-n4c5ccccc5c5ccccc54)c3n2-c2ccccc2)cc1 ; c1ccc(-c2nc3ccc(-n4c5ccccc5c5ccccc54)cc3n2-c2ccccc2)cc1 ; c1ccc(-c2nc3cc(-n4c5ccccc5c5ccccc54)ccc3n2-c2ccccc2)cc1 ; c1ccc(-c2nc3c(-n4c5ccccc5c5ccccc54)cccc3n2-c2ccccc2)cc1]

The electron transporting layer is disposed on the light emitting layer. The second conducting layer is disposed on the electron transporting layer.

In one embodiment of the present invention, the first conducting layer is an anode.

In one embodiment of the present invention, the hole transporting layer includes a hole injection layer and a hole transporting layer disposed on the hole injection layer.

In one embodiment of the present invention, the electron transporting layer includes an electron transporting layer and an electron injection layer disposed on the electron transporting layer.

In one embodiment of the present invention, the second conducting layer is a cathode.

In one embodiment of the present invention, the substrate is a glass substrate.

In one embodiment of the present invention, the substrate is a plastic substrate.

In one embodiment of the present invention, the material of the first conducting layer is indium tin oxide (ITO).

In one embodiment of the present invention, the material of the second conducting layer is aluminum.

### BRIEF DESCRIPTION OF THE DRAWINGS

FIG. 1 is a schematic diagram of an embodiment of the organic light emitting diode of the present invention.

FIG. 2 is a schematic diagram of a different embodiment of the organic light emitting diode of the present invention.

### DETAILED DESCRIPTION OF THE PREFERRED EMBODIMENT

This disclosure introduces Benzimidazole (hereinafter “BImP”) group as an electron-transporting group and Carbazole (hereinafter “Cz”) group as a hole-transporting group, wherein a series of bipolar molecules are synthesized by nitrogen-carbon bonding. The series of bipolar molecules are quite suitable to be the host materials of phosphor organic light emitting diodes (PHOLEDs) due to the relatively fine thermal stability of BImP groups and the higher triplet energy of Cz groups.

Specifically, the bipolar material of the present invention is selected from the group consisting of one of formula (1) to formula (4) or combinations thereof, wherein:

formula (1), hereinafter as “1-CzBImP”;

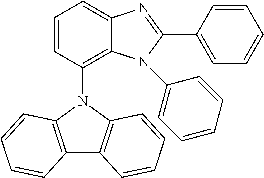 [SMILES: c1ccc(-c2nc3cccc(-n4c5ccccc5c5ccccc54)c3n2-c2ccccc2)cc1]

formula (2), hereinafter as “2-CzBImP”;

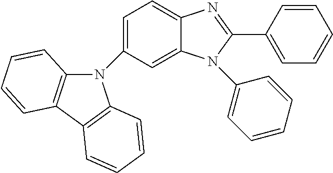 [SMILES: c1ccc(-c2nc3ccc(-n4c5ccccc5c5ccccc54)cc3n2-c2ccccc2)cc1]

formula (3), hereinafter as “3-CzBImP”;

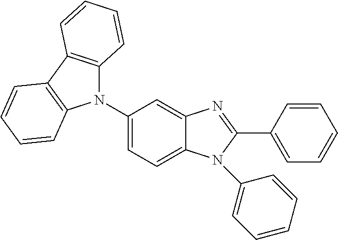 [SMILES: c1ccc(-c2nc3cc(-n4c5ccccc5c5ccccc54)ccc3n2-c2ccccc2)cc1]

formula (4), hereinafter as “4-CzBImP”.

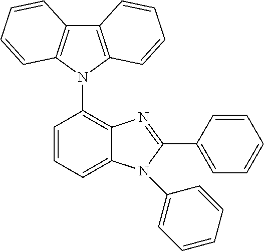 [SMILES: c1ccc(-c2nc3c(-n4c5ccccc5c5ccccc54)cccc3n2-c2ccccc2)cc1]

The compounds of the above four formulas are synthesized by chemical method and characterized by mass spectrometer and element analysis. The results are listed below.

1-CzBImP: ¹H NMR (400 MHz, CD₂Cl₂): 8.06 (d, J=4.2, 1H), 7.85 (d, J=3.9, 2H), 7.54 (t, J=7.8, 1H), 7.41-7.35 (m, 3H), 7.30-7.25 (m, 3H), 7.21-7.11 (m, 4H), 6.92 (d, J=4.1, 2H), 6.65 (t, J=7.5, 1H), 6.47 (d, J=4.2, 2H) 6.34 (t, J=6.9, 2H); ¹³C NMR (100 MHz, CD₂Cl₂): 153.59, 145.45, 141.88, 135.01, 134.16, 129.87, 129.35, 128.00, 127.75, 127.19, 126.61, 125.46, 125.42, 123.21, 122.76, 121.22, 120.80, 119.66, 119.29, 109.74; HRMS calcd for C₃₁H₂₁N₃ (M⁺) 436.1814, obsd. 436.1835. Anal. Calcd for C₃₁H₂₁N₃: C, 85.49; H, 4.86; N, 9.65. Found: C, 85.51; H, 5.07; N, 9.63.

2-CzBImP: ¹H NMR (400 MHz, CD₂Cl₂): 8.15 (d, J=3.9, 2H), 8.06 (d, J=4.2, 1H), 7.66-7.64 (m, 2H), 7.53-7.35 (m, 14H), 7.28 (t, J=7.1, 2H); ¹³C NMR (100 MHz, CD₂Cl₂): 153.76, 142.57, 141.53, 138.24, 136.70, 133.00, 129.99, 129.93, 129.62, 129.48, 128.80, 128.32, 127.35, 125.93, 123.07, 122.58, 120.76, 120.15, 119.74, 109.72, 109.69; HRMS calcd for C₃₁H₂₁N₃(M⁺) 436.1808, obsd. 436.1813. Anal. Calcd for C₃₁H₂₁N₃: C, 85.49; H, 4.86; N, 9.65. Found: C, 85.51; H, 5.02; N, 9.68.

3-CzBImP: ¹H NMR (400 MHz, CD₂Cl₂): 8.18 (d, J=3.9, 2H), 8.02 (s, 1H), 7.65-7.56 (m, 5H), 7.44-7.28 (m, 13H); ¹³C NMR (100 MHz, CD₂Cl₂): 153.77, 143.98, 141.59, 136.88, 132.68, 130.01, 129.93, 129.64, 129.49, 128.87, 128.31, 127.48, 125.91, 123.14, 122.73, 120.15, 119.70, 118.54, 111.52, 109.81; HRMS calcd for C₃₁H₂₁N₃ (M⁺) 436.1808, obsd. 436.1805. Anal. Calcd for C₃₁H₂₁N₃: C, 85.49; H, 4.86; N, 9.65. Found: C, 85.50; H, 4.71; N, 9.63.

4-CzBImP: ¹H NMR (400 MHz, CD₂Cl₂): 8.21 (d, J=3.9, 2H), 7.58-7.22 (m, 19H); ¹³C NMR (100 MHz, CD₂Cl₂): 153.40, 142.02, 140.22, 140.04, 137.48, 130.57, 130.30, 130.18, 130.13, 129.46, 129.03, 128.77, 128.14, 126.28, 124.18, 124.02, 122.22, 120.65, 120.34, 111.40, 110.87; HRMS calcd for C₁₃H₂₁N₃ (M⁺) 436.1808, obsd. 436.1823. Anal. Calcd for C₃₁H₂₁N₃: C, 85.49; H, 4.86; N, 9.65. Found: C, 85.56; H, 4.79; N, 9.60.

The evaluation methods for the host materials is to perform measurements of triplet energy (E_{T}), glass transition temperature (T_{g}), decomposition temperature (T_{d}), highest occupied molecular orbital level (HOMO), and lowest unoccupied molecular orbital level (LUMO) of the above-mentioned compounds, respectively. The triplet energy measured at low temperature by spectrometer is the basis for selecting the host material of phosphorescent luminary. For blue light-emitting diode, FIrpic (E_{T} of 2.65 eV) is a common phosphorescent luminary and the E_{T} of the host luminary developed by FIrpic should be higher than 2.65 eV to avoid back energy transfer which results in reducing luminous efficiency. The glass transition temperature and the decomposition temperature respectively measured by differential scanning calorimeter (DSC) and thermogravimetric analysis (TGA) is considered to be the basis of stability for the fabrication and performance of the unit. HOMOs and LUMOs are acquired receptively from oxidation potential and reduction potential of the materials by using cyclic voltammetry, which can facilitate in the search for an electron injection material with small difference energy gap and enhance the efficiency of the unit.

The properties of the compounds of 1-CbBImP, 2-CbBImP, 3-CbBImP, and 4-CbBImP are shown in Table 1.

TABLE 1

| | Table 1 The properties of the bipolar compounds | | | | |
|---|---|---|---|---|---|
| | E_{T} | T_{g} | T_{d} | HOMO | LUMO |
| | (eV) | (° C.) | (° C.) | (eV) | (eV) |
| 1-CbBImP | 2.92 | 53 | 312 | 5.79 | 2.32 |
| 2-CbBImP | 2.93 | 83 | 354 | 5.72 | 2.35 |
| 3-CbBImP | 2.87 | 86 | 357 | 5.70 | 2.36 |
| 4-CbBImP | 2.88 | 90 | 356 | 5.68 | 2.36 |

According to Table 1, the decomposition temperatures of the compounds are all higher than 300° C. It is because that their structures contain aromatic rings which are rigid structures, so that the decomposition caused by the heat does not easily occur during the heating process. Based on the reason mentioned above, their derivatives have fine thermal stability and high triplet energy and are quite suitable to be the host materials in organic luminescent layer of organic light emitting diodes.

In one embodiment of the present invention, the preparation of the compounds of 1-CbBImP, 2-CbBImP, 3-CbBImP, and 4-CbBImP are shown by the following equations.

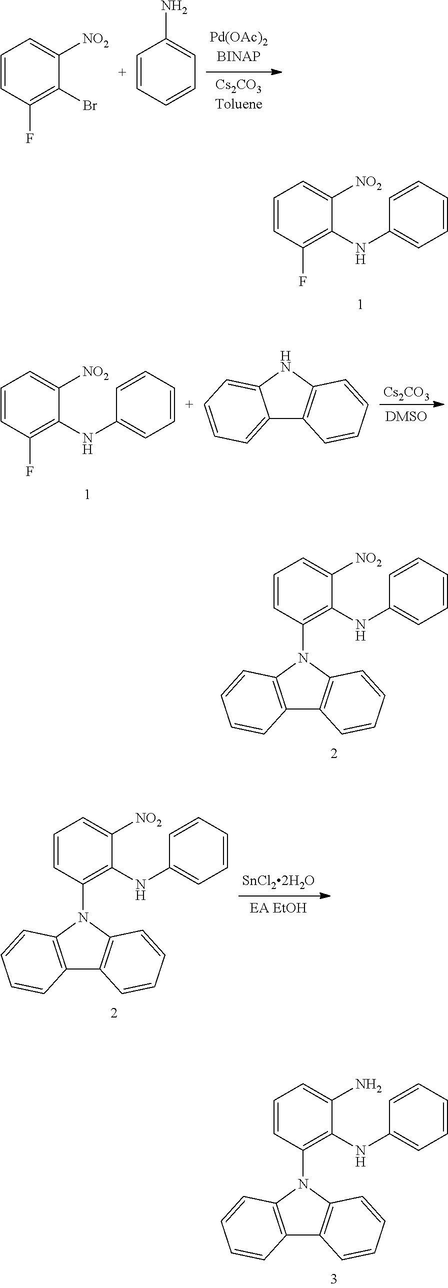

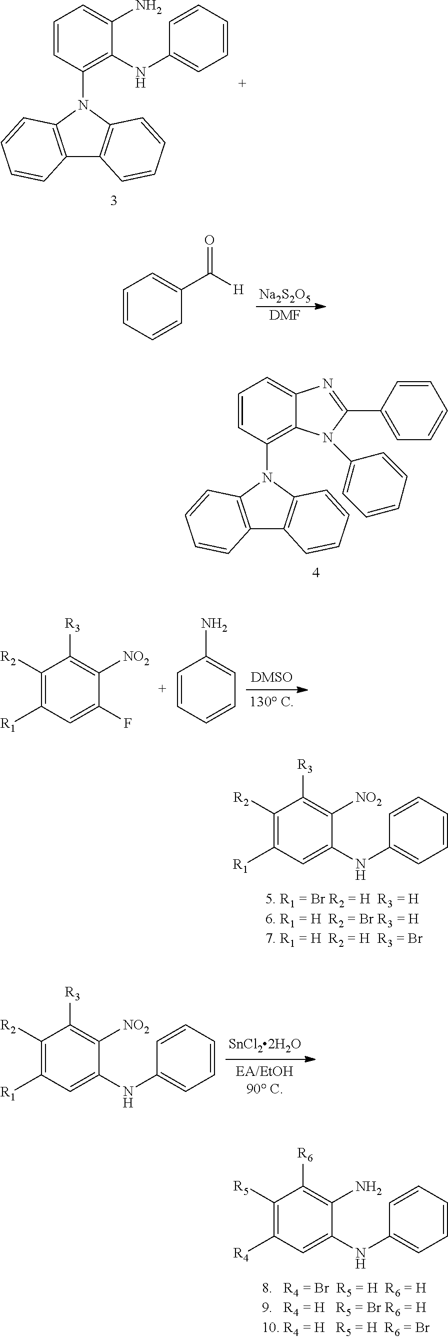

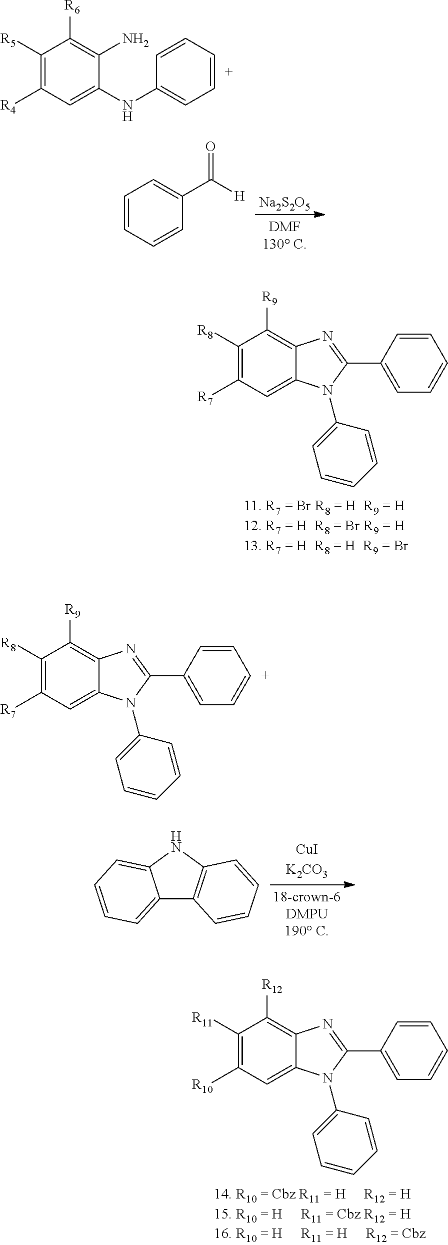

Specifically, in the above equations, N-(2-bromo-6-nitrophenyl)benzenamine of compound 1 is shown as the following formula.

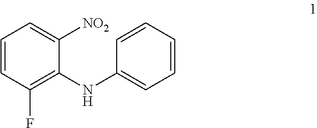 [SMILES: O=[N+]([O-])c1cccc(F)c1Nc1ccccc1]

### Preparation of N-(2-bromo-6-nitrophenyl)benzenamine

2-Bromo-3-fluoronitrobenzene (10.0 g, 45.4 mmol), aniline (4.6 g, 50 mmol), palladium(II) acetate (Pd(OAc)₂, 0.2 g, 0.9 mmol), rac-BINAP((±)-2,2′-Bis(diphenylphosphino)-1,1′-binaphthalene) (BINAP, 0.8 g, 1.4 mmol), Caesium carbonate (45.0 g, 136.4 mmol) were placed into a two neck flask (250 mL). Nitrogen was replaced and toluene (100 mL) was injected. The mixture was refluxed at 115° C. for 8 hours, followed by cooling to room temperature. The mixture was filtered through celite and was dried by vacuum. After the mixture was extracted by ethyl acetate and saline water and purified by column chromatography, 11.0 g of red brown liquid was obtained, and the yield is 96%.

Spectral data are as follows: ¹H NMR (400 MHz, d₆-DMSO): 8.53 (s, 1H), 7.86 (dt, J₁=4.2, J₂=1.4, 1H), 7.60 (dd, J₁=10.8, J₂=4.1, 1H), 7.22-7.17 (m, 3H), 6.90-6.84 (m, 3H); ¹³C NMR (100 MHz, d₆-DMSO): 155.72 (d, J_{CF}=247.5), 142.78, 142.44, 128.75, 126.49, 122.00, 121.50, 121.24 (d, J_{CF}=10.0), 121.02, 116.64.

N-(2-(9H-carbazol-9-yl)-6-nitrophenyl)benzenamine of compound 2 is shown as the following formula.

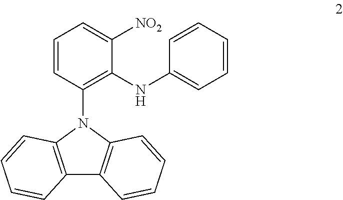 [SMILES: O=[N+]([O-])c1cccc(-n2c3ccccc3c3ccccc32)c1Nc1ccccc1]

### Preparation of N-(2-(9H-carbazol-9-yl)-6-nitrophenyl)benzenamine

Compound 1 (11.0 g, 47.4 mmol), Carbazole (8.7 g, 52.1 mmol), Caesium carbonate (17 g, 52.1 mmol), and DMSO (133 mL) were placed into a single neck flask (250 mL). The mixture was heated to 130° C. for 18 hours, followed by cooling to room temperature. The solvent was removed by vacuum distillation. The mixture was extracted by ethyl acetate and saline water. The organic layer is dehydrated by anhydrous magnesium sulfate, followed by drying by rotary evaporator. After the mixture was purified by column chromatography, dried by rotary evaporator, washed by n-hexane and a little ethyl acetate, and filtered by suction, 3.9 g of red brown solid was obtained, and the yield is 22%.

Spectral data are as follows: m.p. 189-190° C.; ¹H NMR (400 MHz, CD₂Cl₂): 8.80 (s, 1H), 8.34 (d, J=4.9, 1H), 7.85 (d, J=3.9, 1H), 7.74 (d, J=4.9, 1H), 7.42 (t, J=7.8, 2H), 7.27-7.14 (m, 5H), 6.45-6.29 (m, 5H); ¹³C NMR (100 MHz, CD₂Cl₂): 140.10, 139.14, 138.78, 137.74, 137.00, 128.67, 127.03, 125.98, 123.46, 123.34, 120.89, 120.01, 119.84, 119.18, 110.02.

6-(9H-carbazol-9-yl)-N¹-phenylbenzene-1,2-diamine of compound 3 is shown as the following formula.

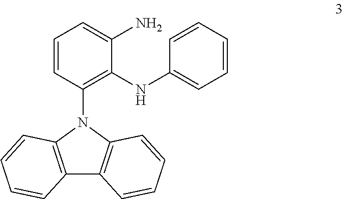 [SMILES: Nc1cccc(-n2c3ccccc3c3ccccc32)c1Nc1ccccc1]

### Preparation of 6-(9H-carbazol-9-yl)-N¹-phenylbenzene-1,2-diamine

Compound 2 (1.7 g, 4.5 mmol), Tin(II) chloride dehydrate (5.1 g, 22.4 mmol), ethanol (15 mL), ethyl acetate (15 mL) were placed into a single neck flask (250 mL). The mixture was refluxed at 90° C. for 5 hours, followed by cooling to room temperature. Potassium hydroxide aqueous solution was added to cause separate layers and extracted by ethyl acetate. The organic layer was dehydrated by anhydrous magnesium sulfate, and dried by rotary evaporator, 1.6 g of light yellow solid was obtained, and the yield is 99%.

Spectral data are as follows: m.p. 171-172° C.; ¹H NMR (400 MHz, d₆-DMSO): 8.05 (d, J=3.8, 2H), 7.29 (t, J=7.5, 2H), 7.18-7.12 (m, 5H), 6.96 (d, J=6.2, 1H), 6.81 (s, 1H), 6.70 (t, J=7.8, 2H), 6.62 (d, J=4.5, 1H), 6.32 (t, J=8.8, 1H), 6.26 (d, J=3.9, 2H), 5.04 (s, 2H); ¹³C NMR (100 MHz, d₆-DMSO): 147.01, 145.54, 140.77, 134.61, 128.47, 126.85, 125.98, 124.09, 123.04, 120.44, 119.63, 117.43, 116.43, 115.14, 113.74, 110.83; HRMS calcd for C₂₄H₁₉N₃ (M⁺) 350.1657, obsd. 350.1640.

9-(1,2-diphenyl-1H-benzo[d]imidazol-7-yl)-9H-carbazole of compound 4 is shown as the following formula.

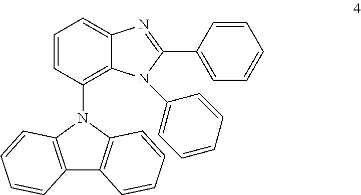 [SMILES: c1ccc(-c2nc3cccc(-n4c5ccccc5c5ccccc54)c3n2-c2ccccc2)cc1]

### Preparation of 9-(1,2-diphenyl-1H-benzo[d]imidazol-7-yl)-9H-carbazole

Compound 3 (1.6 g, 4.6 mmol), Benzaldehyde (0.6 g, 5.1 mmol), Sodium metabisulfite (1.0 g, 5.1 mmol), and Calcium hydride (CaH₂) dehydrated Dimethylformamide (DMF, 25 mL) were took and reacted at 130° C. for 16 hours, followed by cooling to room temperature. The solution was dropped into water and filtered by suction to obtain gray solid. After extracted by dichloromethane and saline water, organic layer was dehydrated by anhydrous magnesium sulfate and dried by rotary evaporator, washed with a little acetone and ethyl acetate, and recrystallized by dichloromethane and ethanol, 1.5 g of white solid was obtained, and the yield is 73%.

Spectral data as follow: m.p. 219-220° C.; ¹H NMR (400 MHz, CD₂Cl₂): 8.06 (d, J=4.2, 1H), 7.85 (d, J=3.9, 2H), 7.54 (t, J=7.8, 1H), 7.41-7.35 (m, 3H), 7.30-7.25 (m, 3H), 7.21-7.11 (m, 4H), 6.92 (d, J=4.1, 2H), 6.65 (t, J=7.5, 1H), 6.47 (d, J=4.2, 2H) 6.34 (t, J=6.9, 2H); ¹³C NMR (100 MHz, CD₂Cl₂): 153.59, 145.45, 141.88, 135.01, 134.16, 129.87, 129.35, 128.00, 127.75, 127.19, 126.61, 125.46, 125.42, 123.21, 122.76, 121.22, 120.80, 119.66, 119.29, 109.74; HRMS calcd for C₃₁H₂₁N₃(M⁺) 436.1814, obsd. 436.1835. Anal. Calcd for C₃₁H₂₁N₃: C, 85.49; H, 4.86; N, 9.65. Found: C, 85.51; H, 5.07; N, 9.63.

N-(5-bromo-2-nitrophenyl)benzenamine of compound 5 is shown as the following formula.

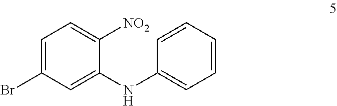 [SMILES: O=[N+]([O-])c1ccc(Br)cc1Nc1ccccc1]

### Preparation of N-(5-bromo-2-nitrophenyl)benzenamine

4-Bromo-2-fluoro-1-nitrobenzene (10 g, 46.1 mmol), aniline (5.2 g, 55.3 mmol), DMSO (130 mL) were took and reacted at 130° C. for 16 hours, followed by cooling to room temperature. The solvent was removed by vacuum distillation. The mixture was extracted by ethyl acetate and saline water. The organic layer was dehydrated by anhydrous magnesium sulfate and dried by rotary evaporator. 12.4 g of red liquid was obtained, and the yield is 92%.

Spectral data as follow: ¹H NMR (400 MHz, d₆-DMSO): 9.49 (s, 1H), 8.04 (d, J=4.5, 1H), 7.46 (t, J=7.6, 2H), 7.35 (d, J=3.9, 2H), 7.27 (t, J=7.3, 1H), 7.16 (s, 1H), 7.00 (d, J=4.5, 1H); ¹³C NMR (100 MHz, d₆-DMSO): 143.15, 138.45, 132.27, 129.94, 129.71, 128.18, 125.80, 124.63, 120.46, 118.27.

N-(4-bromo-2-nitrophenyl)benzenamine of compound 6 is shown as the following formula.

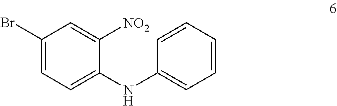 [SMILES: O=[N+]([O-])c1cc(Br)ccc1Nc1ccccc1]

### Preparation of N-(4-bromo-2-nitrophenyl)benzenamine

5-Bromo-2-fluoro-1-nitrobenzene (10 g, 46.1 mmol), aniline (5.2 g, 55.3 mmol), DMSO (130 mL) were took and reacted at 130° C. for 16 hours. The solvent was removed by vacuum distillation. The mixture was extracted by ethyl acetate and saline water. The organic layer was dehydrated by anhydrous magnesium sulfate and dried by rotary evaporator. 13.2 g of red liquid was obtained, and the yield is 98%.

Spectral data as follow: ¹H NMR (400 MHz, d₆-DMSO): 9.41 (s, 1H), 8.19 (d, J=1.2, 1H), 7.59 (dd, J₁=3.4, J₂=2.4, 1H), 7.41 (t, J=7.4, 2H), 7.31-7.29 (m, 2H), 7.22 (t, J=7.4, 1H), 7.09 (d, J=4.6, 1H); ¹³C NMR (100 MHz, d₆-DMSO): 141.87, 139.28, 138.77, 134.24, 130.05, 128.39, 125.80, 124.54, 119.38, 108.23; HRMS calcd for C₁₂H₉BrN₂O₂ (M⁺) 290.9769, obsd. 290.9869.

N-(3-bromo-2-nitrophenyl)benzenamine of compound 7 is shown as the following formula.

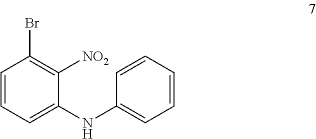 [SMILES: O=[N+]([O-])c1c(Br)cccc1Nc1ccccc1]

### Preparation of N-(3-bromo-2-nitrophenyl)benzenamine

2-Bromo-6-fluoronitrobenzene (3.0 g, 13.6 mmol), aniline (1.5 g, 16.4 mmol), DMSO (38 mL) were took and reacted at 130° C. for 16 hours. The solvent was removed by vacuum distillation. The mixture was extracted by ethyl acetate and saline water. The organic layer was dehydrated by anhydrous magnesium sulfate and dried by rotary evaporator. After purified by column chromatography, 3.1 g of red liquid was obtained, and the yield is 65%. Spectral data as follow: ¹H NMR (400 MHz, d₆-DMSO): 8.21 (s, 1H), 7.34-7.26 (m, 5H), 7.10-7.07 (m, 2H), 7.00 (t, J=8.9, 1H); ¹³C NMR (100 MHz, d₆-DMSO): 142.12, 141.68, 138.10, 132.33, 129.26, 124.35, 122.40, 119.79, 119.09, 113.42; HRMS calcd for C₁₂H₉BrN₂O₂ (M⁺) 290.9769, obsd. 290.9789.

5-bromo-N¹-phenylbenzene-1,2-diamine of compound 8 is shown as the following formula.

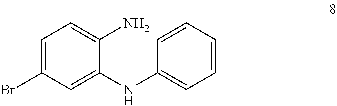 [SMILES: Nc1ccc(Br)cc1Nc1ccccc1]

### Preparation of 5-bromo-N¹-phenylbenzene-1,2-diamine

Compound 5 (12.0 g, 41.1 mmol), Tin(II) chloride dehydrate (45.0 g, 205.5 mmol), ethanol (150 mL), ethyl acetate (150 mL) were took and refluxed at 90° C. for 5 hours, followed by cooling to room temperature. Potassium hydroxide aqueous solution was added to cause separate layers. After extracted by ethyl acetate and dried by rotary evaporator, 9.5 g of light pink solid was obtained, and the yield is 88%.

Spectral data as follow: m.p. 77-78° C.; ¹H NMR (400 MHz, d₆-DMSO): 7.22-7.16 (m, 3H), 7.08 (s, 1H), 6.94 (d, J=5.3, 1H), 6.80-6.68 (m, 4H), 4.94 (s, 2H); ¹³C NMR (100 MHz, d₆-DMSO): 144.78, 141.09, 129.85, 129.02, 125.79, 123.67, 118.76, 116.55, 115.55, 106.41; HRMS calcd for Cl₂H₁₁BrN₂ (M⁺) 263.0184, obsd. 263.0207.

4-bromo-N¹-phenylbenzene-1,2-diamine of compound 9 is shown as the following formula.

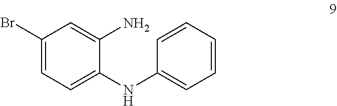 [SMILES: Nc1cc(Br)ccc1Nc1ccccc1]

### Preparation of 4-bromo-N¹-phenylbenzene-1,2-diamine

Compound 6 (14.3 g, 48.8 mmol), Tin(II) chloride dehydrate (55.0 g, 244.0 mmol), ethanol (170 mL), ethyl acetate (170 mL) were took and refluxed at 90° C. for 5 hours, followed by cooling to room temperature. Potassium hydroxide aqueous solution was added to cause separate layers. After extracted by ethyl acetate and dried by rotary evaporator, 1.08 g of light pink solid was obtained, and the yield is 85%.

Spectral data as follow: m.p. 102-103° C.; ¹H NMR (400 MHz, d₆-DMSO): 7.16-7.12 (m, 3H), 6.94-6.91 (m, 2H), 6.76-6.64 (m, 4H), 5.07 (s, 2H); ¹³C NMR (100 MHz, d₆-DMSO): 145.34, 144.00, 128.92, 127.15, 124.16, 118.54, 118.16, 116.91, 115.60, 114.91; HRMS calcd for C₁₂H₁₁BrN₂ (M⁺) 263.0184, obsd. 263.0180.

3-bromo-N¹-phenylbenzene-1,2-diamine of compound 10 is shown as the following formula.

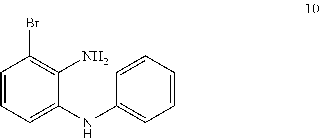 [SMILES: Nc1c(Br)cccc1Nc1ccccc1]

### Preparation of 3-bromo-N¹-phenylbenzene-1,2-diamine

Compound 7 (2.4 g, 8.1 mmol), Tin(II) chloride dehydrate (9.1 g, 40.1 mmol), ethanol (28 mL), ethyl acetate (28 mL) were took and refluxed at 90° C. for 5 hours, followed by cooling to room temperature. Potassium hydroxide aqueous solution was added to cause separate layers. After extracted by ethyl acetate and dried by rotary evaporator, 2.1 g of light pink solid was obtained, and the yield is 97%.

Spectral data as follow: m.p. 77-78° C.; ¹H NMR (400 MHz, d₆-DMSO): 7.33 (s, 1H), 7.18-7.14 (m, 3H), 7.04 (d, J=3.9, 1H), 6.78-6.71 (m, 3H), 6.52 (t, J=7.9, 1H), 4.87 (s, 2H); ¹³C NMR (100 MHz, d₆-DMSO): 145.21, 139.57, 129.37, 128.97, 126.99, 121.77, 118.48, 126.99, 121.77, 118.48, 117.30, 115.15, 108.41; HRMS calcd for C₁₂H₁₁BrN₂ (M⁺) 263.0184, obsd. 263.0190.

6-bromo-1,2-diphenyl-1H-benzo[d]imidazole of compound 11 is shown as the following formula.

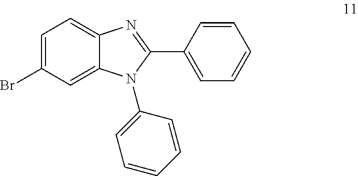 [SMILES: Brc1ccc2nc(-c3ccccc3)n(-c3ccccc3)c2c1]

### Preparation of 6-bromo-1,2-diphenyl-1H-benzo[d]imidazole

Compound 8 (9.5 g, 36.1 mmol), Benzaldehyde (4.2 g, 39.7 mmol), Sodium metabisulfite (4.3 g, 39.7 mmol), and Calcium hydride (CaH₂) dehydrated Dimethylformamide (DMF, 195 mL) were took and reacted at 130° C. for 16 hours, followed by cooling to room temperature. The solution was dropped into water and filtered to obtain brown solid. After washed with ether, ethanol and a little ethyl acetate, and recrystallized by dichloromethane/ethanol, 10.6 g of light yellow solid was obtained, and the yield is 87%. Spectral data as follow: m.p. 161-162° C.; ¹H NMR (400 MHz, d₆-DMSO): 7.77 (d, J=4.3, 1H), 7.59-7.35 (m, 11H), 7.30 (d, J=0.9, 1H); ¹³C NMR (100 MHz, d₆-DMSO): 153.24, 142.13, 138.70, 136.40, 130.25, 129.78, 129.60, 128.84, 127.94, 126.22, 121.64, 116.04, 113.55; HRMS calcd for C₁₉H₁₃BrN₂ (M⁺) 349.0340, obsd. 349.0330.

5-bromo-1,2-diphenyl-1H-benzo[d]imidazole of compound 12 is shown as the following formula.

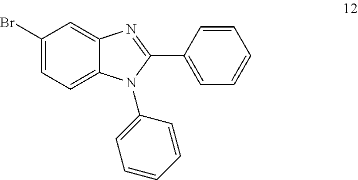 [SMILES: Brc1ccc2c(c1)nc(-c1ccccc1)n2-c1ccccc1]

### Preparation of 5-bromo-1,2-diphenyl-1H-benzo[d]imidazole

Compound 9 (10.2 g, 38.9 mmol), Benzaldehyde (4.5 g, 42.8 mmol), Sodium metabisulfite (7.45 g, 39.3 mmol), and Calcium hydride (CaH₂) dehydrated Dimethylformamide (DMF, 200 mL) were took and reacted at 130° C. for 16 hours, followed by cooling to room temperature. The solution was dropped into water and filtered to obtain brown solid. After washed with ether, ethanol and a little ethyl acetate, and recrystallized by dichloromethane/ethanol, 9.4 g of light yellow solid was obtained, and the yield is 72%.

Spectral data as follow: m.p. 168-169° C.; ¹H NMR (400 MHz, d₆-DMSO): 8.01 (d, J=0.92, 1H), 7.61-7.51 (m, 5H), 7.45-7.35 (m, 6H), 7.15 (d, J=4.3, 1 H); 13C NMR (100 MHz, d₆-DMSO): 153.55, 144.35, 136.71, 130.56, 130.33, 129.76, 129.64, 129.60, 128.87, 127.95, 126.50, 122.20, 115.31, 112.82; HRMS calcd for C₁₉H₁₃BrN₂ (M⁺) 349.0340, obsd. 349.0365.

4-bromo-1,2-diphenyl-1H-benzo[d]imidazole of compound 13 is shown as the following formula.

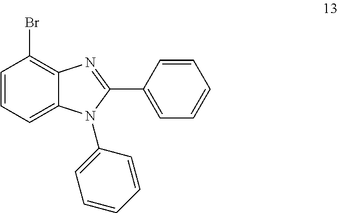 [SMILES: Brc1cccc2c1nc(-c1ccccc1)n2-c1ccccc1]

### Preparation of 4-bromo-1,2-diphenyl-1H-benzo[d]imidazole

Compound 10 (2.1 g, 7.8 mmol), Benzaldehyde (0.9 g, 8.6 mmol), Sodium metabisulfite (1.5 g, 8.0 mmol), and Calcium hydride (CaH₂) dehydrated Dimethylformamide (DMF, 42 mL) were took and reacted at 130° C. for 16 hours, followed by cooling to room temperature. The solution was dropped into water and filtered to obtain brown solid. After washed with ether, ethanol and a little ethyl acetate, and recrystallized by dichloromethane/ethanol, 2.0 g of light yellow solid was obtained, and the yield is 78%.

Spectral data as follow: m.p. 151-152° C.; ¹H NMR (400 MHz, d₆-DMSO): 7.60-7.37 (m, 11H), 7.24-7.18 (m, 2H); ¹³C NMR (100 MHz, d₆-DMSO): 153.08, 141.43, 138.18, 136.60, 130.55, 130.34, 129.79, 129.75, 129.68, 128.86, 128.06, 126.02, 125.02, 112.81, 110.70; HRMS calcd for C₁₉H₁₃BrN₂ (M⁺) 349.0340, obsd. 349.0332.

9-(1,2-diphenyl-1H-benzo[d]imidazol-6-yl)-9H-carbazole of compound 14 is shown as the following formula.

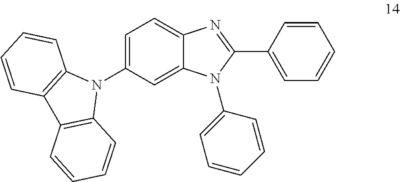 [SMILES: c1ccc(-c2nc3ccc(-n4c5ccccc5c5ccccc54)cc3n2-c2ccccc2)cc1]

### Preparation of 9-(1,2-diphenyl-1H-benzo[d]imidazol-6-yl)-9H-carbazole

Compound 11 (9.9 g, 29.3 mmol), Carbazole (5.6 g, 33.1 mmol), Copper(I) iodide (Cu(I)I) (0.2 g, 0.9 mmol), 1,4,7,10,13,16-hexaoxacyclooctadecane (18-crown-6) (0.2 g, 0.9 mmol), Potassium carbonate (K₂CO₃) (20.3 g, 146.5 mmol) 1,3-Dimethyl-3,4,5,6-tetrahydro-2(1H)-pyrimidinone (DMPU) (20 mL) were took and heated to 130° C., and reacted for 48 hours. The solvent was removed by vacuum distillation. The mixture was filtered through celite, washed with dichloromethane and dried by vacuum. After extracted by dichloromethane and saline water, washed with a little acetone and ethyl acetate, and dried by rotary evaporator, gray solid was obtained. After purified by column chromatography, recrystallized by dichloromethane/ethanol, and sublimation, 9.7 g of white solid was obtained, and the yield is 76%.

Spectral data as follow: m.p. 192-193 ¹³C; ¹H NMR (400 MHz, CD₂Cl₂): 8.15 (d, J=3.9, 2H), 8.06 (d, J=4.2, 1H), 7.66-7.64 (m, 2H), 7.53-7.35 (m, 14H), 7.28 (t, J=7.1, 2H); ¹³C NMR (100 MHz, CD₂Cl₂): 153.76, 142.57, 141.53, 138.24, 136.70, 133.00, 129.99, 129.93, 129.62, 129.48, 128.80, 128.32, 127.35, 125.93, 123.07, 122.58, 120.76, 120.15, 119.74, 109.72, 109.69; HRMS calcd for C₃₁H₂₁N₃ (M+) 436.1808, obsd. 436.1813. Anal. Calcd for C₃₁H₂₁N₃: C, 85.49; H, 4.86; N, 9.65. Found: C, 85.51: H, 5.02; N, 9.68.

9-(1,2-diphenyl-1H-benzo[d]imidazol-5-yl)-9H-carbazole of compound 15 is shown as the following formula.

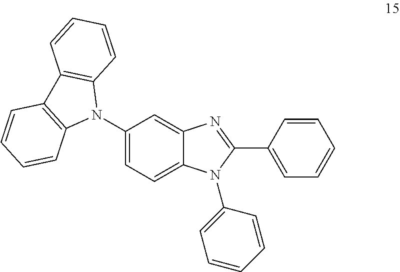 [SMILES: c1ccc(-c2nc3cc(-n4c5ccccc5c5ccccc54)ccc3n2-c2ccccc2)cc1]

### Preparation of 9-(1,2-diphenyl-1H-benzo[d]imidazol-5-yl)-9H-carbazole

Compound 12 (7.8 g, 23.2 mmol), Carbazole (4.4 g, 26.2 mmol), Copper(I) iodide (Cu(I)I) (0.1 g, 0.7 mmol), 1,4,7,10,13,16-hexaoxacyclooctadecane (18-crown-6) (0.2 g, 0.7 mmol), Potassium carbonate (K₂CO₃) (16.0 g, 115.8 mmol) 1,3-Dimethyl-3,4,5,6-tetrahydro-2(1H)-pyrimidinone (DMPU) (15 mL) were took and heated to 200° C., and reacted for 48 hours. The solvent was removed by vacuum distillation. The mixture was filtered through celite, washed with dichloromethane and dried by vacuum. After extracted by dichloromethane and saline water, washed with acetone and ethyl acetate, and dried by rotary evaporator, gray solid was obtained. After purified by column chromatography, recrystallized by dichloromethane/ethanol, and sublimation, 7.2 g of white solid was obtained, and the yield is 72%.

Spectral data as follow: m.p. 235-236 ¹³C; ¹H NMR (400 MHz, CD₂Cl₂): 8.18 (d, J=3.9, 2H), 8.02 (s, 1H), 7.65-7.56 (m, 5H), 7.44-7.28 (m, 13H); ¹³C NMR (100 MHz, CD₂Cl₂): 153.77, 143.98, 141.59, 136.88, 132.68, 130.01, 129.93, 129.64, 129.49, 128.87, 128.31, 127.48, 125.91, 123.14, 122.73, 120.15, 119.70, 118.54, 111.52, 109.81; HRMS calcd for C₃₁H₂₁N₃(M⁺) 436.1808, obsd. 436.1805. Anal. Calcd for C₃₁H₂₁N₃: C, 85.49; H, 4.86; N, 9.65. Found: C, 85.50; H, 4.71; N, 9.63.

9-(1,2-diphenyl-1H-benzo[d]imidazol-4-yl)-9H-carbazole of compound 16 is shown as the following formula.

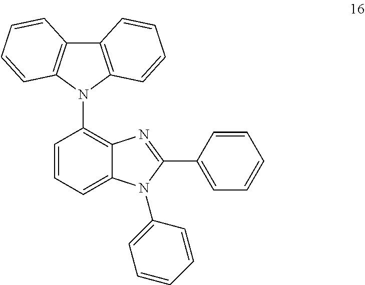 [SMILES: c1ccc(-c2nc3c(-n4c5ccccc5c5ccccc54)cccc3n2-c2ccccc2)cc1]

### Preparation of 9-(1,2-diphenyl-1H-benzo[d]imidazol-4-yl)-9H-carbazole

Compound 13 (2.7 g, 7.9 mmol), Carbazole (1.5 g, 8.7 mmol), Copper(I) iodide (Cu(I)I) (0.05 g, 0.24 mmol), 1,4,7,10,13,16-hexaoxacyclooctadecane (18-crown-6) (0.06 g, 0.24 mmol), Potassium carbonate (K₂CO₃) (5.5 g, 39.6 mmol) 1,3-Dimethyl-3,4,5,6-tetrahydro-2(1H)-pyrimidinone (DMPU) (5 mL) were took and heated to 200° C., and reacted for 48 hours. The solvent was removed by vacuum distillation. The mixture was filtered through celite, washed with dichloromethane and dried by vacuum. After extracted by dichloromethane and saline water, washed with acetone and ethyl acetate, and dried by rotary evaporator, gray solid was obtained. After purified by column chromatography, recrystallized by dichloromethane/ethanol, and sublimation, 2.4 g of white solid was obtained, and the yield is 71%.

Spectral data as follow: m.p. 274-275° C.; ¹H NMR (400 MHz, CD₂Cl₂): 8.21 (d, J=3.9, 2H), 7.58-7.22 (m, 19H); ¹³C NMR (100 MHz, CD₂Cl₂): 153.40, 142.02, 140.22, 140.04, 137.48, 130.57, 130.30, 130.18, 130.13, 129.46, 129.03, 128.77, 128.14, 126.28, 124.18, 124.02, 122.22, 120.65, 120.34, 111.40, 110.87; HRMS calcd for C₃₁H₂₁N₃(M⁺) 436.1808, obsd. 436.1822. Anal. Calcd for C₃₁H₂₁N₃: C, 85.49; H, 4.86; N, 9.65. Found: C, 85.56; H, 4.79; N, 9.60.

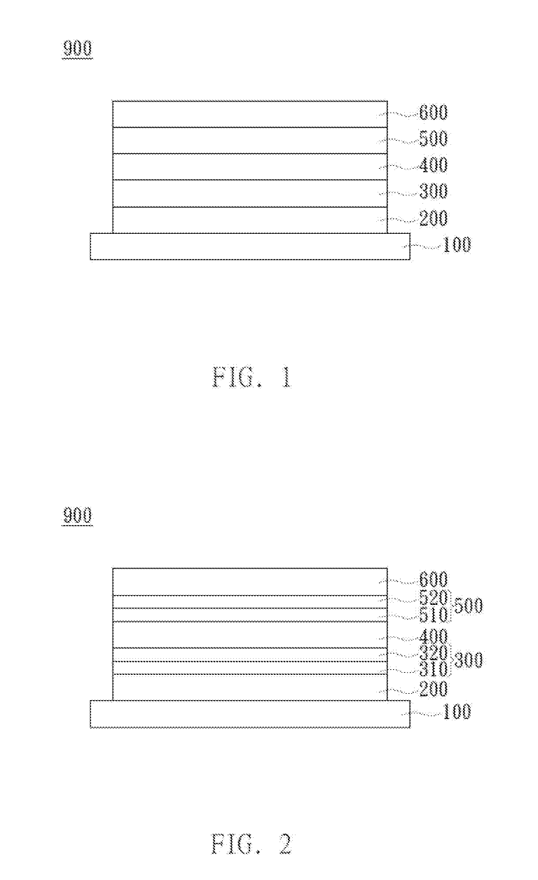

As shown in the embodiment in FIG. 1, the organic light emitting diode 900 of the present invention includes a substrate 100, a first conducting layer 200, a hole transporting layer 300, a light emitting layer 400, an electron transporting layer 500, and a second conducting layer 600. The first conducting layer 200 is disposed on the substrate 100. The hole transporting layer 300 is disposed on the first conducting layer 200. The light emitting layer 400 is disposed on the hole transporting layer 300. The light emitting layer 400 includes a bipolar material selected from the group consisting of one of formula (1) to formula (4) or combinations thereof shown as below. The electron transporting layer 500 is disposed on the light emitting layer 400. The second conducting layer 600 is disposed on the electron transporting layer 500.

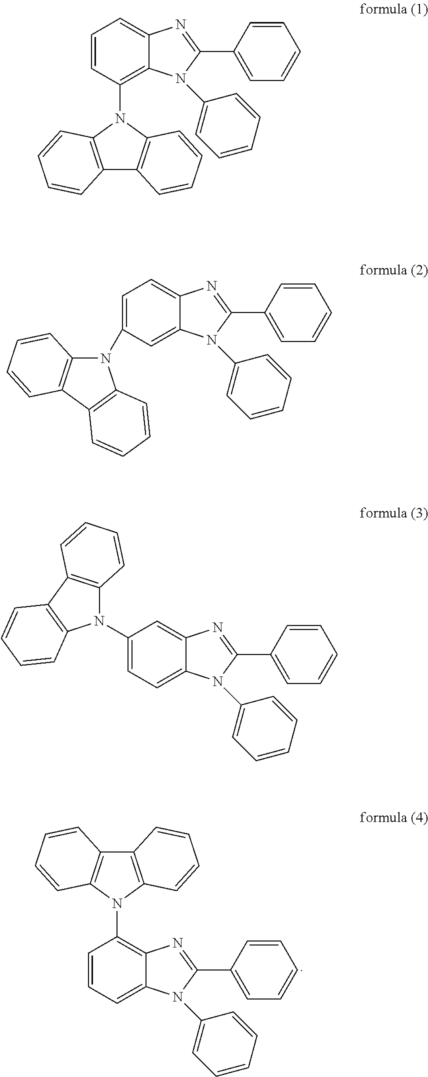 [SMILES: c1ccc(-c2nc3cccc(-n4c5ccccc5c5ccccc54)c3n2-c2ccccc2)cc1 ; c1ccc(-c2nc3ccc(-n4c5ccccc5c5ccccc54)cc3n2-c2ccccc2)cc1 ; c1ccc(-c2nc3cc(-n4c5ccccc5c5ccccc54)ccc3n2-c2ccccc2)cc1 ; c1ccc(-c2nc3c(-n4c5ccccc5c5ccccc54)cccc3n2-c2ccccc2)cc1]

In one embodiment of the present invention, the substrate 100 could be a glass substrate, a plastic substrate, etc. The substrate could be photo-permeable, and further could be transparent. In one embodiment of the present invention, the first conducting layer 200 is an anode, wherein the first conducting layer 200 has a work function preferably greater than 4.5 eV. The material of the first conducting layer could be indium tin oxide (ITO), SnO₂, Au, Ag, Pt, or Cu, etc. No special limit is set to the material of the hole transporting layer 300 in the present invention. Compounds which are generally used as the material of the hole transporting layer are acceptable, e.g. 4,4′-Cyclohexyl idenebis[N,N-bis(4-methylphenyl)benzenamine] (TAPC), 1,3-Bis(N-carbazolyl)benzene (mCP), N,N′-Bis(3-methylphenyl)-N,N′-diphenylbenzidine (TPD), or α-naphylhenyldiamine (NPB).

No special limit is set to the material of the electron transporting layer 500 in the present invention. Compounds which are generally used as the material of the electron transporting layer are acceptable, e.g. Diphenylbis(4-(pyridin-3-yl)phenyl)silane (DPPS), LiF, AlQ3, Bebq2 (Bis(10-hydroxybenzo[h]quinolinato)beryllium). 3-(Biphenyl-4-yl)-5-(4-tert-butylphenyl)-4-phenyl-4H-1,2,4-triazole (TAZ), or 2,9-Dimethyl-4,7-diphenyl-1,10-phenanthroline (BCP). The second conducting layer 600 is a cathode, wherein the second conducting layer 600 has a smaller work function. The material of the second conducting layer 600 could be indium, Al, In—Mg alloy, Magnalium, Al—Li alloy, or Mg—Ag alloy.

As shown in a different embodiment in FIG. 2, the hole transporting layer 300 includes a hole injection layer 310 and a hole transporting layer 320 disposed on the hole injection layer 310. The electron transporting layer 500 includes an electron transporting layer 510 and an electron injection layer 520 disposed on the electron transporting layer 510.

In one embodiment, the organic light emitting diode is manufactured by an evaporation process, wherein the structure of the organic light emitting diode is first conducting layer (ITO)/hole injection layer (TAPC, 50 nm)/hole transporting layer (mCP, 10 nm)/light emitting layer (30 nm)/electron transporting layer (DPPS, 45 nm)/electron injection layer (LiF, 0.7 nm)/second conducting layer (Al, 120 nm). The light emitting layer takes the prepared 2-CbBImP, 3-CbBImP, and 4-CbBImP as the host materials and uses different doping ratios of emitter (e.g. FIrpic (Bis[2-(4,6-difluorophenyl)pyridinato-C²,N](picolinato)iridium(III)) as the guest material. The driving voltage (V) under the current density of 20 mA/cm², the maximum current efficiency (cd/A), the maximum power efficiency (lm/W), and the maximum external quantum efficiency (EQE) (%) of the organic light emitting diodes are measured.

The results are shown in Table 2.

TABLE 2

| | | maximum | | |
|---|---|---|---|---|
| | driving | current | power | |
| | voltage | efficiency | efficiency | |
| Unit^{a} | (V)^{b} | (cd/A) | (lm/W) | EQEₘₐₓ(%) |
| 2-CbBImP-18% | 6.56 | 44.07 | 39.63 | 20.42 |
| 3-CbBImP-15% | 7.07 | 50.25 | 45.10 | 23.95 |
| 4-CbBImP-15% | 6.91 | 53.25 | 47.89 | 24.82 |

^{a}The doping concentration of FIrpic
^{b}the unit of operation voltage under 20 mA/cm²

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A bipolar material selected from the group consisting of one of formula (1) to formula (4) or combinations thereof, wherein:

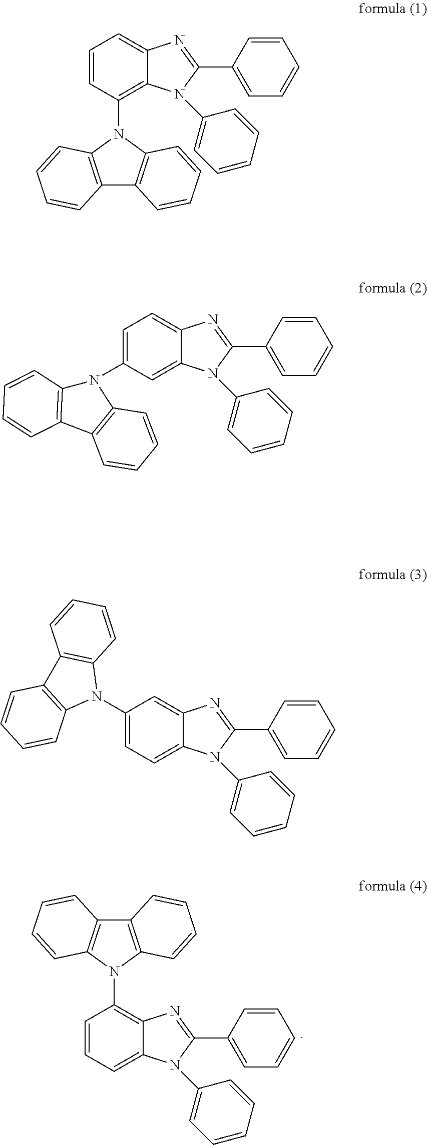 [SMILES: c1ccc(-c2nc3cccc(-n4c5ccccc5c5ccccc54)c3n2-c2ccccc2)cc1 ; c1ccc(-c2nc3ccc(-n4c5ccccc5c5ccccc54)cc3n2-c2ccccc2)cc1 ; c1ccc(-c2nc3cc(-n4c5ccccc5c5ccccc54)ccc3n2-c2ccccc2)cc1 ; c1ccc(-c2nc3c(-n4c5ccccc5c5ccccc54)cccc3n2-c2ccccc2)cc1]

2. An organic light emitting diode, comprising:
a substrate;
a first conducting layer disposed on the substrate;
a hole transporting layer disposed on the first conducting layer;
a light emitting layer disposed on the hole transporting layer, wherein the light emitting layer includes a bipolar material selected from the group consisting of one of formula (1) to formula (4) or combinations thereof, wherein: 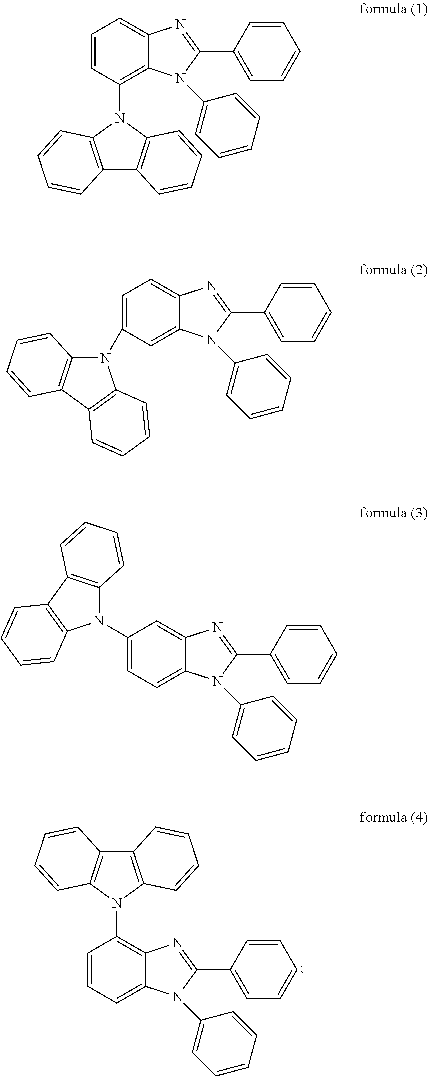 [SMILES: c1ccc(-c2nc3cccc(-n4c5ccccc5c5ccccc54)c3n2-c2ccccc2)cc1 ; c1ccc(-c2nc3ccc(-n4c5ccccc5c5ccccc54)cc3n2-c2ccccc2)cc1 ; c1ccc(-c2nc3cc(-n4c5ccccc5c5ccccc54)ccc3n2-c2ccccc2)cc1 ; c1ccc(-c2nc3c(-n4c5ccccc5c5ccccc54)cccc3n2-c2ccccc2)cc1]
an electron transporting layer disposed on the light emitting layer; and
a second conducting layer disposed on the electron transporting layer.

3. The organic light emitting diode of claim 2, wherein the first conducting layer is an anode.

4. The organic light emitting diode of claim 2, wherein the hole transporting layer includes a hole injection layer and a hole transporting layer disposed on the hole injection layer.

5. The organic light emitting diode of claim 2, wherein the electron transporting layer includes an electron transporting layer and an electron injection layer disposed on the electron transporting layer.

6. The organic light emitting diode of claim 2, wherein the second conducting layer is a cathode.

7. The organic light emitting diode of claim 2, wherein the substrate is a glass substrate.

8. The organic light emitting diode of claim 2, wherein the substrate is a plastic substrate.

9. The organic light emitting diode of claim 2, wherein the material of the first conducting layer is indium tin oxide (ITO).

10. The organic light emitting diode of claim 2, wherein the material of the second conducting layer is aluminum.
